(19) United States
(12) Patent Application Publication
(10) Pub. No.: US 2021/0236858 A1
(43) Pub. Date: Aug. 5, 2021

(54) AUTOMATED ULTRASOUND BLEEDING DETECTION AND TREATMENT

(71) Applicant: General Electric Company, Schenectady, NY (US)

(72) Inventors: Lowell Scott Smith, Schenectady, NY (US); Luca Marinelli, Schenectady, NY (US); Larry Y.L. Mo, Latham, NY (US); Antonio Caiafa, Albany, NY (US); David Shoudy, Niskayuna, NY (US)

(21) Appl. No.: 16/781,823

(22) Filed: Feb. 4, 2020

(51) Int. Cl. A61N 7/00; A61B 5/02; A61B 8/06; A61B 8/00; A61N 7/02; A61B 8/08

(52) U.S. Cl. CPC A61N 7/00; A61B 5/02042; A61B 8/06; A61B 8/4461; A61B 8/4494; A61N 7/02; A61N 2007/0078; A61B 8/5223; A61B 8/461; A61N 2007/0052; A61N 2007/0082; A61B 8/467; A61N 2007/0017; A61B 8/5207

(57) ABSTRACT

In accordance with the present disclosure, ultrasound-based techniques using a combined scanning and treatment array module are employed to find and treat anomalies corresponding to bleed events. By way of example, ultrasound data may be acquired with a scanning array at one or more locations on a patient anatomy. A treatment array may deliver heat to a targeted anomaly to provide therapy. Such a technique may be useful outside of a hospital environment.

### TECHNICAL FIELD

The subject matter disclosed herein relates to detection and treatment of bleeding, including identification and treatment of such occurrences outside of a hospital environment.

### BACKGROUND

Vascular trauma with vessel disruption can occur in a variety of environments, including both military and civilian environments. In some instances, the vascular trauma may be internal, without a clear break (e.g., an entry or exit wound) in the skin corresponding to the location of the trauma. In such circumstances it may be difficult to identify where in the body an internal bleeding event is occurring and provide treatment, or, indeed, if there is internal bleeding occurring at all.

For example, a skilled or trained person may be able to determine if a bleed event is present based on indications of vascular injury that include pulsatile hemorrhage, expanding hematoma, bruit or thrill over the injury site, absent extremity pulses, and arterial pressure index <0.9. However, such indications may be insufficient to make such a determination even by a trained individual, and likely would be impossible or impractical for an untrained individual to evaluate. Further, even to the extent these factors may allow a skilled or trained person to determine if a vascular injury is present, they may be still insufficiently skilled to localize the internal site of the vascular trauma, which is necessary to apply treatment to a patient.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

In one embodiment, an ultrasound based scanning and treatment system is provided. In accordance with this embodiment, the ultrasound based scanning and treatment system comprises: an ultrasound probe, a motor, and a processor. The ultrasound probe comprises a first treatment array, a second treatment array and a scanning array. The scanning array is configured to generate a set of image data for an imaged volume. The first treatment array and second treatment array are configured to deliver heat to a portion of the imaged volume. The motor is configured to incrementally move the ultrasound probe. The processor is communicatively coupled to the ultrasound probe and the motor and configured to determine a location of an anomaly in the set of image data, wherein the portion of the imaged volume contains the anomaly.

In a further embodiment, a method is provided for detecting and treating bleeding events. In accordance with this method, a set of ultrasound image data is acquired while incrementally moving a scanning array in an elevation direction relative to a scanned volume. The set of ultrasound image data is used to detect an anomaly. A treatment array of the ultrasound probe targets the anomaly and delivers energy to the targeted anomaly.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates a schematic diagram of an embodiment of an ultrasound-based scanning and treatment system, in accordance with aspects of the present disclosure;

FIG. 2 illustrates a device incorporating an ultrasound-based scanning and treatment system, in accordance with aspects of the present disclosure;

FIG. 3 illustrates associated movement axes and orientations of an ultrasound probe, in accordance with aspects of the present disclosure;

FIGS. 4A and 4B illustrate a dual-mode array module of an ultrasound-based scanning and treatment system from a front view (FIG. 4A) and side view (FIG. 4B), in accordance with aspects of the present disclosure; and

FIG. 5 illustrates a process flow of an ultrasound-based scanning and treatment process, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The present disclosure relates to the automatic detection and treatment of bleeding events. Based on the derived bleed location, feedback can be provided to a treatment array. More generally, once the bleed has been detected and accurately localized, therapy to contain blood loss can be delivered. Detailed information about the location of the bleed would enable deployment of therapy in a location determined to maximize therapeutic effectiveness and to minimize side effects. For example, high-intensity focused ultrasound (HIFU) may be employed to cauterize a bleed site, with automatic steering of the HIFU beam being accomplished using the bleed location as determined by the techniques discussed herein.

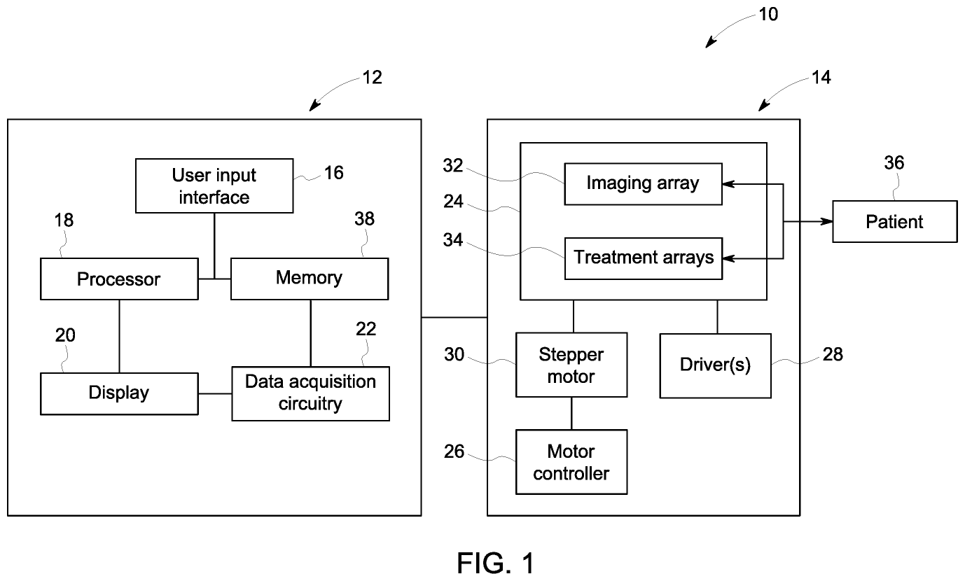

With the preceding comments in mind, FIG. 1 illustrates a schematic diagram of an embodiment of an ultrasound-based scanning and treatment system 10 that may be used to identify and treat bleeding, as described herein. The ultrasound-based scanning and treatment system 10 may include a system controller block 12 and an array module 14. The system controller block 12 may control operation of the array module 14 and may process image data acquired by the array module 14. The array module 14 may be coupled to the system controller block 12 by any suitable techniques for communicating image data and control signals between the array module 14 and the system controller block 12 such as a wireless, optical, coaxial, or other suitable connection.

The array module 14 may include an ultrasound probe 24, a motor controller 26, one or more drivers 28, and a stepper motor 30. The ultrasound probe 24 contacts the patient 36 during an ultrasound examination. The ultrasound probe 24 may include a patient facing or contacting surface, a scanning array 32, and a plurality of treatment arrays 34. The scanning array 32 may include a transducer element capable of operating in a switched manner between transmit and receive modes. The scanning array 32 may be capable of converting electrical energy into mechanical energy for transmission and mechanical energy into electrical energy for receiving. It should be noted that the scanning array 32 may be configured as a two-way array capable of transmitting ultrasound waves into and receiving such energy from a subject or patient 36 during operation when the ultrasound probe 24 is placed in contact with the patient 36. More specifically, the scanning array 32 may convert electrical energy from the ultrasound probe 24 into ultrasound waves (e.g., ultrasound energy, acoustic waves) and transmit the ultrasound waves into the patient 36. The ultrasound waves may be reflected back toward the scanning array 32, such as from tissue of the patient 36, and the scanning array 32 may convert the ultrasound energy received from the patient 36 (reflected signals or echoes) into electrical signals for transmission and processing by the array module 14 and system controller block 12. The scanning array 32 may scan a 2-dimensional plane in the patient 36 to generate scanning data. The scanning array 32 may generate a set of scanning data corresponding to each of the scanned 2-dimensional planes in the patient.

The stepper motor 30 may be capable of moving the ultrasound probe 24 by incremental steps in a direction substantially orthogonal to the scanned 2-dimensional plane. For example, the direction may be within fifteen degrees of orthogonal. In certain embodiments, the stepper motor 30 may be capable of moving the ultrasound probe 24 by incremental steps in a direction non-parallel to the scanned 2-dimensional plane. In some embodiments, the stepper motor 30 may be capable of moving the ultrasound probe 24 by incremental steps in non-parallel to a scanning plane of the scanning array 32. The scanning array 32 may generate a sequential set of ultrasound beams at each incremental step. The motor controller 26 may be configured to control operation of the stepper motor 30.

Each of the plurality of treatment arrays 34 may include a transducer element capable of providing energy (e.g., heat based cauterization where the heat is generated using high-intensity focused ultrasound (HIFU)) to a bleeding location to reduce or stop the flow of blood. One or more drivers 28 may be configured to apply a desired voltage level to a corresponding treatment array 34. The one or more drivers 28 may be configured to generate a sequence of pulses at a desired frequency during a treatment mode of the system 10. In certain embodiments, the treatment arrays 34 may be phased treatment arrays. For example, the emitted HIFU from the treatment arrays 34 may be electronically steered by adjusting phases of the energy emitted from the treatment arrays 34. The emitted HIFU from the treatment arrays 34 constructively interfere to increase the amount of energy (e.g., heat based cauterization where the heat is generated using HIFU) delivered in a desired direction towards a bleeding location.

As will be appreciated, the system controller block 12 may include a number of elements to control operation of the array module 14, facilitate placement/guidance of the array module 14, and facilitate production and/or interpretation of ultrasound images. For instance, as illustrated, the system controller block 12 may include a user input interface 16, a processor 18, a display 20, data acquisition circuitry 22, and memory 38. In certain embodiments, the system controller block 12 may include additional elements not shown in FIG. 1 such as additional data acquisition and processing controls, additional display panels, multiple user interfaces, and so forth.

The user input interface 16 may be capable of receiving an input from a user to begin the scanning mode, the treatment mode, or any combination thereof. The user input interface 16 may be capable of receiving an input from a user to decline or terminate a scanning mode, a treatment mode, or any combination thereof. The user input interface 16 may be capable of receiving an input from a user to begin a suggested treatment mode after detection of a bleeding location. The user input interface 16 may be a portion of the display 20. For example, the user input interface 16 may be a touch screen. The display 20 may provide an indication of a current operating mode of the system 10. The display 20 may also provide an indication that the system 10 suggests performing a treatment mode after detection of a bleeding location. The display 20 may also provide an indication that a user manually move the system 10 to a new location on the patient to perform a scanning mode, a treatment mode, or any combination thereof. In some embodiments, the user input interface 16 may include a set of buttons. For example, the user input interface 16 may include a start scan button, a start treatment button, a cancel button, or any combination thereof.

The data acquisition circuitry 22 may be communicatively coupled to the processor 18. The data acquisition circuitry 22 may include receiving and conversion circuitry. The data acquisition circuitry 22 may receive the set of scanning data from the array module 14 representing reflected ultrasound energy returned from tissue interfaces within the patient 36. The data acquisition circuitry 22 may process the data from the array module 14, such as correcting for noise artifacts, time-gain compensation, beamforming, or the like. The data acquisition circuitry 22 may generate a set of scanned two-dimensional image data (i.e., unreconstructed image data) corresponding to each of the scanned two dimensional planes in the patient. The data acquisition circuitry 22 may transmit the scanned two dimensional image data to the processor 18.

Based on a first input received at the user input interface 16, the processor 18 may output a signal to the ultrasound probe 24 to transmit ultrasound waves from the scanning array 32 into the patient 36 and to subsequently detect at the scanning array 32 the ultrasound energy that is reflected back from the tissue interfaces within the patient 36. Based on a second input received at the user input interface and/or in response to detection and localization of a bleeding site, the processor 18 may output a signal to the drivers 28 and also to the ultrasound probe 24 indicative of an instruction to transmit from the treatment arrays 34 high-intensity focused ultrasound capable of generating heat within the target tissue of the patient 36 to provide treatment to the identified bleeding site.

In some embodiments, the memory 38 may include one or more tangible, non-transitory, computer-readable media that store instructions executable by the processor 18 and/or data to be processed by the processor 18. For example, the memory 38 may include random access memory (RAM), read only memory (ROM), rewritable non-volatile memory such as flash memory, hard drives, optical discs, and/or the like. Additionally, the processor may include one or more general purpose microprocessors, one or more application specific processors (ASICs), one or more field programmable logic arrays (FPGAs), or any combination thereof. Further, the memory 38 may store scan data obtained via the array module 14 and/or algorithms utilized by the processor 18 to help guide and/or activate the treatment arrays 34 based on bleed localization information generated based on data acquired using the scanning array 32. The processor 18 may control transmission of the ultrasound waves into the patient 36 via the ultrasound probe 24. Additionally, the processor 18 may process acquired data to generate a sequence of ultrasound images, may construct a three dimensional image from such a sequence of images, and/or may detect and localize a bleeding site.

The processor 18 may receive scanning data and/or the scanned two dimensional image data from the data acquisition circuitry 22. The scanning data and/or scanned two dimensional image data may correspond to a sequence of two dimensional scanned planes in the patient 36. The processor 18 may process the scanning data and/or scanned two dimensional image data to determine flow rates and/or flow directions for liquids within the patient 36. For example, the processor may use Doppler scanning to determine flow rates and directions by calculating frequency shifts from a set of ultrasound waves reflecting off a volume of fluid. Additionally, the processor 18 may process the scanning data and/or scanned two dimensional image data to construct a three dimensional image. For example, the sequence of two dimensional scanned planes may correspond to a series of incremental movements of the ultrasound probe 24 in an elevation direction. The processor 18 may construct a three dimensional image by sorting the sequence of two dimensional scanned planes in order based on a corresponding elevation of each scanned plane. In certain embodiments, the system 10 may include a sensor for providing the corresponding elevation of each scanned plane. In some embodiments, a motor control signal may provide the corresponding elevation of each scanned plane. The processor 18 may detect and localize a bleeding site based on the scanning data and/or sampled two dimensional image data (i.e., unreconstructed image data) and/or the constructed three dimensional image. The processor 18 may compare generated and/or constructed images of the patient 36 to previous healthy images stored in memory 38. For example, the processor 18 may determine whether any anomalies are present in the generated and/or constructed images based on previous image data or other reference data sets. The processor 18 may combine flow rates and directions with generated two-dimensional images and/or three-dimensional constructions to determine the location of any bleeding sites.

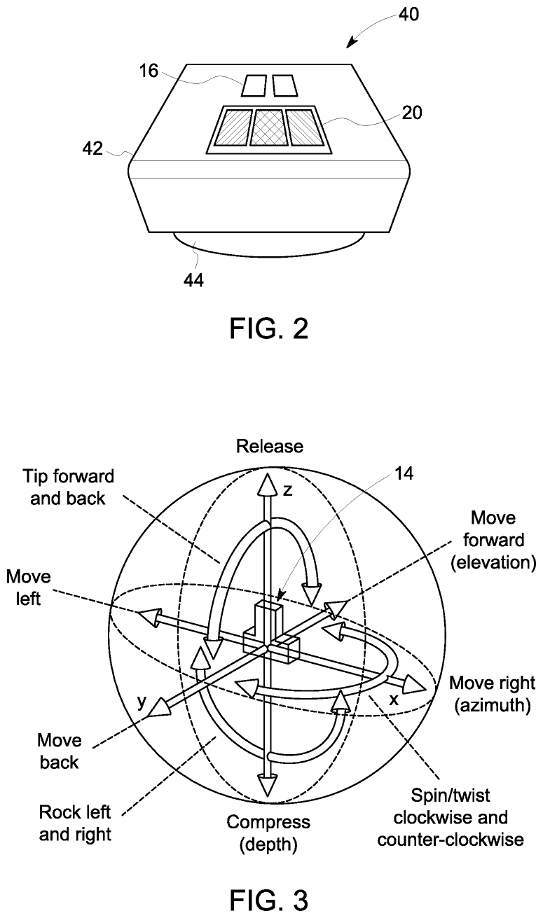

With the preceding in mind, and turning to FIG. 2, an example of a device 40 incorporating and enclosing an ultrasound-based scanning and treatment system, such as system 10 in FIG. 1, is illustrated. In one embodiment, the device 40 may have a total volume equal to or less than thirty two cubic inches, though larger devices 40 are also contemplated. The device 40 includes a user input interface 16, a display 20, a housing 42, and a patient facing or contacting surface 44. In certain embodiments, the patient facing or contacting surface 44 may include a protective shell. The protective shell may be filled with an acoustic coupling medium. The user input interface 16 may include a set of buttons configured to receive an input from a user. The display 20 may include one or more lights and/or an indication on a touch screen display configured to display an operating mode of the device 40. The housing 42 may contain at least one of the components of the system controller block 12 of FIG. 1. The housing 42 may also contain at least one of the components of the array module 14 of FIG. 1.

To facilitate discussion related to motion of an ultrasound probe 24, FIG. 3 illustrates degrees of freedom and axes of motion with respect to an array module, as used herein. As shown in FIG. 3, the three axes of motion (and corresponding degrees of freedom) may be denoted as (elevation (e.g., moving the probe head backward and forward on the patient), azimuth (e.g., moving the probe head from left to right on the patient), compression (moving the probe head downward (compression) and upward (release) on the patient). These axes also may be used in describing three different motions related to probe head rotation or orientation with respect to the patient, which equate to three additional degrees of freedom: tipping (e.g., holding the probe head in place while moving the handle backward and forward), rocking (e.g., holding the probe head in place while moving the handle left and right), and spinning or twisting the probe (e.g., clockwise or counter-clockwise rotation) about an axis of rotation generally corresponding to axis defined by the probe handle.

With this relative motion nomenclature in mind, the present approach allows for ultrasound-based scanning and treatment to determine the existence of bleeding sites and provide treatment to a patient.

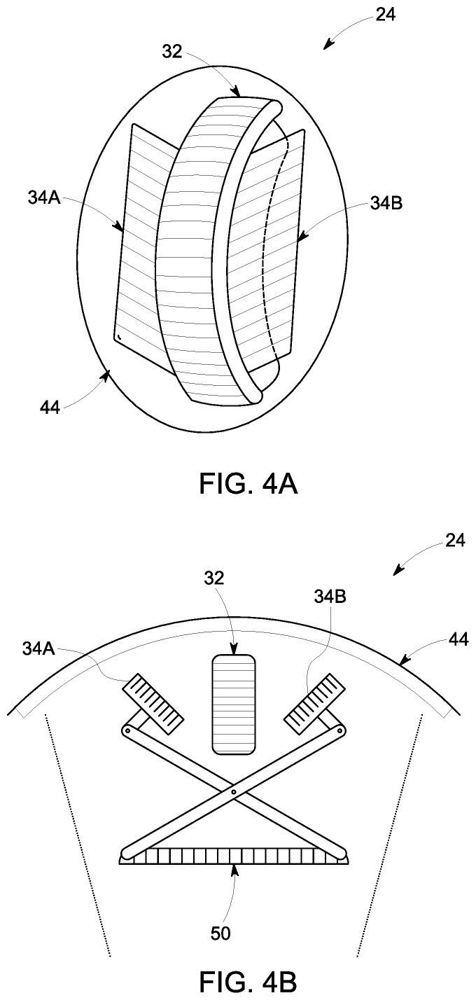

With the preceding context in mind, and turning to FIG. 4A and FIG. 4B, an ultrasound probe, such as ultrasound probe 24 of FIG. 1, of an ultrasound-based scanning and treatment system is illustrated. The ultrasound probe 24 may be moved by a stepper motor, such as stepper motor 30 of FIG. 1, in an elevation direction. The ultrasound probe 24 includes scanning array 32, treatment arrays 34A, 34B, a patient facing or contacting surface 44, and a tilt control assembly 50. The scanning array 32 may be a convex array. In one embodiment, the scanning array 32 may operate with a center frequency between 2.5 to 3.5 MHz. The scanning array 32 may operate with a transmit pulse length of about one microsecond in one such embodiment. The scanning array 32 may scan across an arc length in the azimuthal direction to interrogate a two dimensional image plane. The scanning array 32 may be incrementally tipped, in an elevation direction, by the stepper motor 30 of FIG. 1 to scan a series of two dimensional image planes.

The treatment arrays 34A, 34B may be High Intensity Focused Ultrasound (HIFU) arrays. The treatment arrays 34A, 34B may operate with a frequency between 0.5 to 2.5 MHz in one such implementation. The treatment arrays 34A, 34B may operate with a pulse length of between about 1 and 500 seconds in such an implementation. In certain embodiments, the pulses from the treatment arrays 34 may be modulated. In some embodiments, the pulses from the treatment arrays 34 may operate at less than 100 percent duty cycle. The treatment arrays 34A, 34B may be incrementally tipped about the azimuth, in an elevation direction, by the stepper motor 30 of FIG. 1 to steer the HIFU beams in the elevation directions. In certain embodiments, the treatment arrays 34A, 34B may be incrementally rotated to rotate a treatment plane about the depth axis. The treatment arrays 34A, 34B may be coupled by tilt control assembly 50. The tilt control assembly 50 may be capable of ensuring the treatment arrays 34A, 34B are kept in alignment with each other and scanning array 32. The tilt control assembly 50 may be driven by a stepper motor, such as stepper motor 30 of FIG. 1. In certain embodiments, the tilt control assembly 50 may be driven by a separate motor than the stepper motor 30. The tilt control assembly 50 may operate to focus the treatment arrays 34A, 34B, such that the intersection of the HIFU beams emitted by the treatment arrays 34A, 34B occurs at the appropriate depth, as determined by the location of the bleed site.

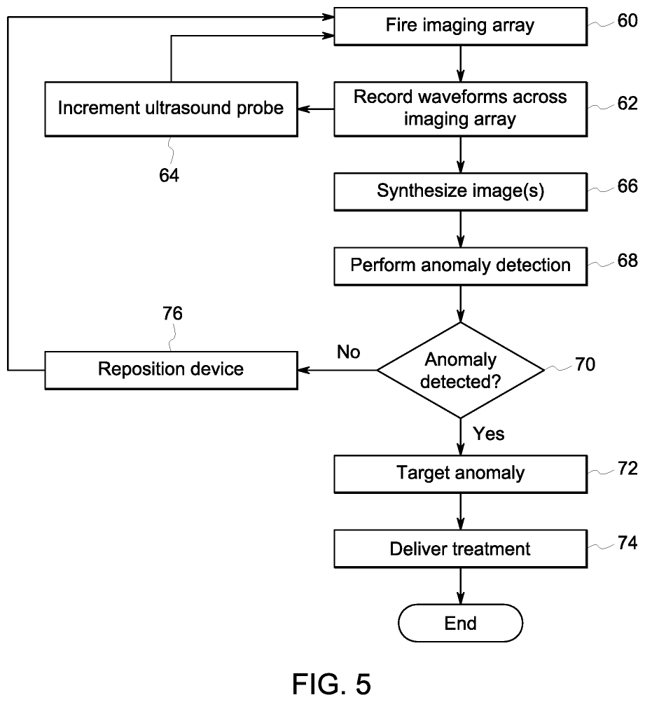

With the preceding in mind, and turning to FIG. 5, a process flow of an ultrasound-based scanning and treatment process is illustrated. In this flow, a user input is received, for example at user input interface 16 of FIG. 1. In response to the user input, the scanning array 32 may be switched between transmitting and receiving modes (denoted as firing scanning array shown at block 60 of FIG. 5) so that ultrasound waves are generated into the tissue and then bounce back or reflect from boundary regions or layers and are subsequently received at the scanning array 32. The received signals may be acquired and/or recorded (step 62) as waveforms across the scanning array 32 by data acquisition circuitry 22. The received signals may correspond to an interrogation of a two dimensional plane of the patient 36. The stepper motor 30 of FIG. 1 may then incrementally move the tipped angle of (step 64) the ultrasound probe 24 to scan another two dimensional plane of the patient 36. The scanning array 32 may generate another sequence of ultrasound waves and receive reflections which are acquired and/or recorded as waveforms across the scanning array 32.

The scanning array 32 may produce a set of recorded waveforms, each recorded waveform corresponding to an incremental movement of the ultrasound probe 24. The set of recorded waveforms may be used to synthesize (step 66) a set of two-dimensional anatomical and/or flow images of the scanned two-dimensional planes of the patient 36. The set of two-dimensional images may be used to construct a three-dimensional image of a scanned volume. At least one image of the set of two-dimensional images and/or the constructed three-dimensional image may be used to perform anomaly detection (step 68) in the scanned volume. For example, the processor 18 may compare scanned, constructed, and/or synthesized images to an image of healthy and/or normal vascular structure to determine whether structural changes corresponding to an anomaly are present.

At step 70, if an anomaly is detected, the processor 18 instructs the array module 14 to target the anomaly (step 72). For example, the processor 18 may instruct the stepper motor 30 to steer the HIFU beams in the azimuthal and/or the elevation direction and to adjust the tilt control assembly 50 to focus the HIFU beams in the elevation direction. In some embodiments, the processor 18 may instruct a separate motor from the stepper motor 30 to adjust the tilt control assembly 50. In response to targeting the anomaly, the treatment arrays generate HIFU beams to deliver energy (e.g., heat) to the targeted anomaly (step 74).

If no anomaly is detected, the display 18 may instruct a user of the device 40 to reposition the device in a new location. The display 18 may then instruct a user to repeat the process of FIG. 5 at the new location.

At step 78, the processor 18 may determine whether a total therapy time has elapsed. In certain embodiments, the total therapy time may be a pulse length of the treatment arrays. If the processor 18 determines the total therapy time has elapsed, the processor 18 may instruct the treatment arrays to end treatment and the process may end. If the processor 18 determines the total therapy time has not elapsed, the process may return to step 60 to generate a subsequent set of ultrasound waves and acquire and/or record reflections at the scanning array 32. The process may continue to determine whether the anomaly has shifted locations relative to the ultrasound-based scanning and treatment system. In certain embodiments, the processor 18 may determine whether a threshold therapy time has elapsed before generating the subsequent set of ultrasound waves. In certain embodiments, the threshold therapy time may be a portion of the pulse length of the treatment arrays. For example, the threshold therapy time may be between five percent and twenty five percent of the pulse length of the treatment arrays.

Technical effects of the invention include, but are not limited to, ultrasound-based detection and treatment of bleeding events. The detection and treatment of these bleeding events may be performed outside of a hospital environment.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound scanning and treatment system, comprising:
an ultrasound probe, comprising:
a scanning array configured to generate a set of scan data for a scanned volume;
a first treatment array and a second treatment array, wherein the first treatment array and second treatment array are configured to deliver a treatment to a portion of the scanned volume;
a motor configured to incrementally move the ultrasound probe; and
a processor communicatively coupled to the ultrasound probe and the motor and configured to determine a location of an anomaly in the set of scan data, wherein the portion of the scanned volume contains the anomaly.

2. The ultrasound scanning and treatment system of claim 1, wherein the first treatment array and the second treatment array are configured to generate high intensity focused ultrasound as part of the treatment.

3. The ultrasound scanning and treatment system of claim 1, wherein the processor is configured to generate a sequence of images from the set of scan data.

4. The ultrasound scanning and treatment system of claim 3, wherein the processor is configured to construct a three dimensional image from the generated sequence of images.

5. The ultrasound scanning and treatment system of claim 1, wherein the processor is configured to determine a set of velocities, flow rates and/or a set of flow directions from the set of scan data.

6. The ultrasound scanning and treatment system of claim 1, wherein the motor is configured to move the scanning array, the first treatment array, and the second treatment array in an elevation direction.

7. The ultrasound scanning and treatment system of claim 1, further comprising a tilt control assembly configured to focus high intensity focused ultrasound beams emitted by the first treatment array and the second treatment array.

8. The ultrasound scanning and treatment system of claim 1, wherein:
the scanning array operates at a first frequency,
the first treatment array and the second treatment array operate at a second frequency, and
the first frequency is greater than the second frequency.

9. The ultrasound scanning and treatment system of claim 1, wherein the first treatment array and the second treatment array operate with a pulse length between 1 and 500 seconds.

10. The ultrasound scanning and treatment system of claim 1, wherein the scanning array operates with a frequency between 2.5-3.5 MHz.

11. The ultrasound scanning and treatment system of claim 1, wherein the first treatment array and the second treatment array operate with a frequency between 0.5-2.5 MHz.

12. The ultrasound scanning and treatment system of claim 1, wherein the anomaly is a bleeding site.

13. The ultrasound scanning and treatment system of claim 1, further comprising a display configured to indicate an operating mode of the ultrasound probe.

14. The ultrasound scanning and treatment system of claim 1, wherein the processor is configured to calculate frequency shifts from a set of ultrasound waves.

15. A method of detecting and treating bleeding events, comprising:
acquiring a set of ultrasound scan data while incrementally moving a scanning array of an ultrasound probe in an elevation direction relative to a scanned volume;
detecting an anomaly in the set of ultrasound scan data;
targeting, in response to detecting the anomaly, the anomaly with a treatment array of the ultrasound probe; and
delivering energy to the targeted anomaly with the treatment array.

16. The method of claim 15, wherein the set of ultrasound scan data corresponds to a set of two dimensional scanned planes in the scanned volume.

17. The method of claim 16, further comprising constructing a three-dimensional image of the scanned volume.

18. The method of claim 15, further comprising determining a set of flow rates and/or a set of flow directions from the set of ultrasound scan data.

19. The method of claim 15, further comprising:
positioning the ultrasound probe relative to a second volume; and
acquiring a second set of ultrasound scan data while incrementally moving the scanning array in the elevation direction relative to the second volume.

20. The method of claim 15, wherein the anomaly is a bleeding site.
